(19) United States
(12) Patent Application Publication
(10) Pub. No.: US 2026/0335788 A1
(43) Pub. Date: Oct. 29, 2026

(54) SELF-SUPPORTING PANEL FOR SIMULATED MOVING BED SEPARATION

(71) Applicant: IFP ENERGIES NOUVELLES, Rueil-Malmaison (FR)

(72) Inventors: Guillaume BLANCKE, Rueil-Malmaison Cedex (FR); Amir Hossein AHMADI-MOTLAGH, Rueil-Malmaison Cedex (FR); Alexandre VONNER, Rueil-Malmaison Cedex (FR); Fabian LAMBERT, Rueil-Malmaison (FR); Clément RAVANAS, Rueil-Malmaison (FR); Daniel HERREMAN, Rueil-Malmaison (FR); Mohamed DAHRAOUI, Rueil-Malmaison (FR); Abdelilah FAIDOLI, Rueil-Malmaison (FR)

(73) Assignee: IFP ENERGIES NOUVELLES, Rueil-Malmaison (FR)

(21) Appl. No.: 19/120,484

(22) PCT Filed: Sep. 29, 2023
(86) PCT No.: PCT/EP2023/077100

§ 371 Date: Apr. 11, 2025

(30) Foreign Application Priority Data
Oct. 12, 2022 (FR) FR2210446

(51) Int. Cl. B01D 15/18; C07C 7/12

(52) U.S. Cl. CPC B01D 15/1842; C07C 7/12

(57) ABSTRACT

Simulated moving bed SMB separation column/process comprising/using a shell (1) comprising a plurality of beds of adsorbent separated by a plurality of plates, each plate comprising a plurality of panels (6) referred to as self-supported panels, each panel (6) comprising a metal frame (7) on multiple sides, wherein the metal frame (7) is supported on a first side (13) by the shell and is supported on a second side (14) by: a main beam (3) disposed diametrically in the shell (1); and/or the shell (1) or a central mast (5) disposed in the shell (1), wherein the metal frame (7) comprises at least one third side (15) connecting the first side (13) to the second side (14), and wherein the at least one third side (15) has a height (H) and a thickness which are suitable for providing the mechanical strength of the panel.

### TECHNICAL FIELD

The invention relates to the field of the separation of natural or chemical products that are difficult to separate by distillation. Use is then made of a family of processes, and of associated devices, known as simulated moving bed separation processes or devices, either by means of simulated countercurrent or simulated cocurrently, which will be referred to below as “SMB”.

The fields concerned are notably the separation of para-xylene from other C8 aromatic isomers. Other fields concerned, non-exclusively, are: the separation of normal paraffins from branched paraffins, naphthenes and aromatics; olefin/paraffin separation; the separation of meta-xylene from other C8 aromatic isomers; the separation of ethylbenzene from other C8 aromatic isomers; the separation of m-cresol or p-cresol from other cresol isomers.

Specifically, the invention concerns a device for distributing and collecting fluids within a multistage column utilizing a flow of said fluids in a medium of solid particles, referred to as bed of adsorbent comprising a granular medium (adsorbent).

Multistage column is understood to mean a column comprising a plurality of beds of adsorbent that are disposed in series in a direction of flow of the one or more fluids utilized in the column. The fluid passing successively through the beds of adsorbent is referred to as main fluid to distinguish it from other, secondary fluids that can be added to the main fluid via a distributing and collecting device, also referred to as plate, which is generally situated between two successive beds (perpendicularly to the direction of flow of the main fluid).

A plate comprises at least one collection zone and a distributing and collecting system (system of lines and valves) for collecting main fluid and/or injecting secondary fluids and mixing these secondary fluids with the main fluid. A plate also comprises at least one distribution zone which has the objective of spreading the fluid resulting from the mixing of the main fluid and the secondary fluids out across the granular bed situated immediately downstream in the direction of flow of the main fluid.

### PRIOR ART

Numerous devices are known for distributing, mixing or collecting a fluid in a chamber containing solid particles, such as in particular a multistage column. The functions of the plates are generally to distribute a fluid as homogeneously as possible over the cross section of the column, to effectively mix the main fluid passing through the various beds of the column with one or more secondary fluids introduced at each bed, optionally to collect a flow of fluid between two beds, and finally to homogenize the concentrations at the bed outlet before entry into the subsequent bed of solid particles, that is to say the bed situated immediately downstream of the device in question, to the best possible extent.

In addition, the plates have to satisfy a certain number of constraints such as generating as little axial dispersion as possible, creating the smallest possible pressure drop, and not producing hydrodynamic disturbances that can have an adverse effect on the performance of the process.

The plates have a certain number of features familiar to those skilled in the art.

For the sake of clarity of the text, a multistage column is divided into a plurality of plates Pi and beds of adsorbent Ai, the plate Pi being disposed directly upstream of the bed of adsorbent Ai in the direction of flow of the main fluid. Furthermore, reference is made to a bed of adsorbent Ai+1 to denote the subsequent bed of adsorbent situated downstream of the bed of adsorbent Ai in the direction of flow of the main fluid. In the same way, a plate Pi+1 denotes the subsequent plate situated downstream of the plate Pi in the direction of flow of the main fluid.

Moreover, each plate Pi of the column is generally divided into multiple sectors or regions, referred to as panels. Each panel of the plate comprises a zone for collecting the main fluid and at least one valve for collecting main fluid and/or injecting secondary fluids and mixing these secondary fluids with the main fluid. Each panel also comprises a distribution zone which has the objective of spreading the fluid resulting from the mixing of the main fluid and the secondary fluids out across the granular bed situated immediately downstream in the direction of flow of the main fluid.

Each panel can have various shapes, the most common being to divide it into angular sectors or meridian panels, that is to say (mutually) parallel panels, substantially of the same width. The current technology generally comprises 12 to 16 panels per plate Pi. EP0074815, US2006/0108274A1, and FR2708480 provide examples of plates used in the case of SMB adsorption.

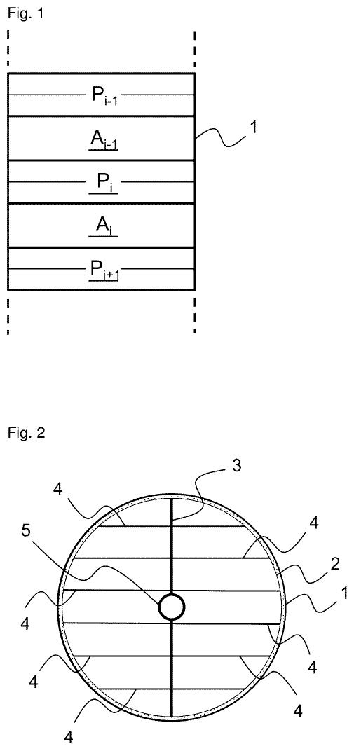

With reference to FIG. 1, the multistage column comprises a shell 1, in which a plurality of beds of solid particles are disposed in series in a direction of flow of a main fluid utilized in the column. Specifically, the shell 1 has the following in the direction of flow of the main fluid: a plate Pi−1, a bed of adsorbent Ai−1 (referred to as upstream bed of adsorbent Ai−1), a plate Pi, a bed of adsorbent Ai (referred to as downstream bed of adsorbent Ai), and a plate Pi+1.

With reference to FIG. 2, the mechanical strength of the plates Pi is provided by one or more weir plates 2 disposed radially (perpendicularly to the direction of flow of the main fluid) on the internal wall of the shell 1 and a network of beams of two different types:
at least one main beam 3 that connects two radially opposite sides of the shell 1 (disposed perpendicularly to the direction of flow of the main fluid) and is designed to support secondary beams 4;
the secondary beams 4 (disposed perpendicularly to the direction of flow of the main fluid), oriented perpendicularly to the main beams that support the panels; and
optionally, a central mast 5 (disposed parallel to the direction of flow of the main fluid, for example vertical mast, that is to say mounted axially in the shell 1), for example in the form of a tube.

The central mast 5 generally serves as a bearing point for the main beam 3 and may comprise one or more lines of the distribution and collection network.

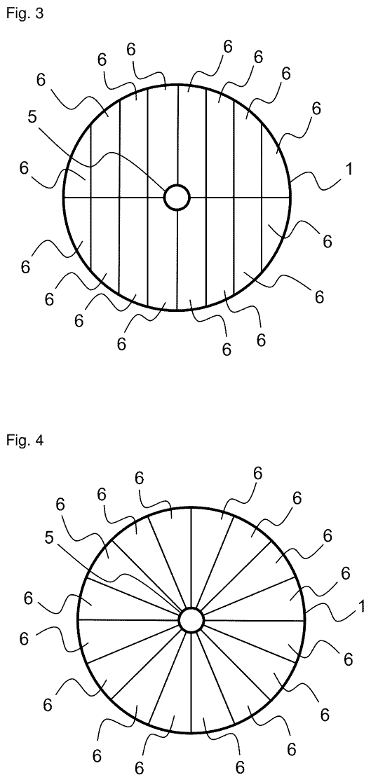

With reference to FIG. 3, the panels 6 may be disposed on the secondary beams 4 in a meridian cutout, for example in 2 parallel rows of 3 or 4 panels.

With reference to FIG. 4, the panels 6 may be disposed on the secondary beams 4 in a radial cutout, for example around the central mast 5.

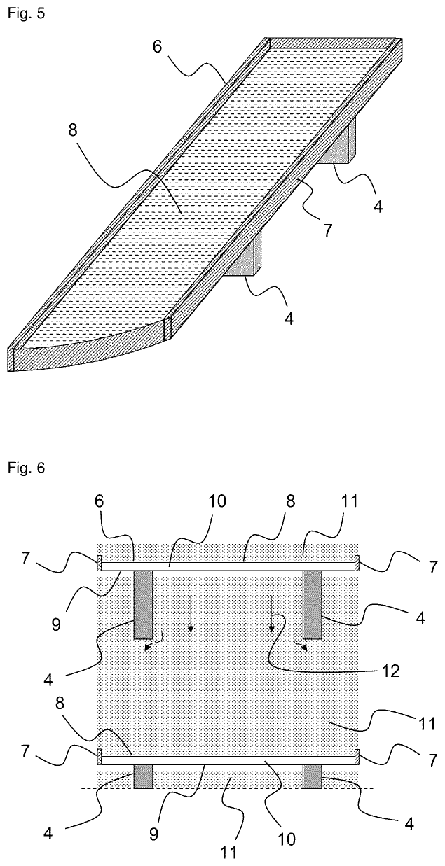

With reference to FIG. 5 and FIG. 6, the secondary beams 4 make it possible to support:
the panels 6 of a plate Pi; and
the bed of adsorbent Ai−1 (for a flow from top to bottom) disposed on the plate Pi.

With reference to FIG. 5 and FIG. 6, each panel 6 of a plate comprises a metal frame 7 which serves to keep the elements of the panel 6 together (screens and inserts), an upper screen 8 and a lower screen 9 for ensuring the passage of the main fluid, and distributing and collecting inserts 10 situated between said screens. The distributing and collecting inserts 10 typically serve to:
collect main fluid from a bed of adsorbent 11 by way of a system referred to as collecting baffle or collector;
mix main fluid that leaves the bed of adsorbent 11 with a secondary fluid optionally injected in the panel in question via a distribution and collection network, generally terminating in an injection-withdrawal tank; and
redistribute the main fluid, collected on its own or in a mixture with a secondary fluid, across the subsequent bed of adsorbent 11 via a distributor.

A multistage column generally has a diameter between 5 and 10 m, this meaning that a panel 6 has a possible length of 5 m and rests against the shell 1 only on one side. Since, to avoid crushing or even breakage of the sieve of the bed of adsorbent 11 situated underneath, the panels 6 should not deform excessively, the mechanical strength for preventing this deformation is provided by the secondary beams 4, which provide a deflection of less than 10 mm irrespective of the location. However, the secondary beams 4 reduce the hydrodynamics in the bed of adsorbent 11, since the flow 12 of main fluid is disrupted (adverse effect on the plug-flow nature). For the one part, it is disadvantageous for the main fluid to make contact with obstacles, such as the secondary beams 4, at the top of the bed. For the other part, the surface area contacted by the main fluid is increased by the secondary beams 4.

FR2961112 describes a self-supported panel in order to reduce the number of obstacles in the bed of adsorbent, the beams being positioned within the distributor panel itself, that is to say between the upper screen and the lower screen. However, the positioning of the beams within the panels themselves hinders the circulation of fluid inside the panel and reduces the distribution quality.

### SUMMARY OF THE INVENTION

Within the context described above, a first object of the present invention is to improve the flow of fluids during SMB separation inside a multistage column, that is to say a column having a multiplicity of beds of adsorbent disposed in series in the direction of flow of the fluids. According to a second object, the present invention makes it possible to reduce the mounting time and to reduce the mass of metal to be utilized for the construction of the multistage column. According to a third object, the present invention makes it possible to improve the homogeneity with which the multistage column is charged with granular medium.

According to a first aspect, the objects mentioned above, and other advantages, are obtained by a simulated moving bed separation column comprising a shell comprising a plurality of beds of adsorbent separated by a plurality of plates, each plate comprising a plurality of panels referred to as self-supported panels, each panel being designed to collect a main fluid from an upstream bed of adsorbent and to supply the main fluid to a downstream bed of adsorbent, each panel comprising a metal frame on multiple sides, the following being disposed in the frame:
an upper screen designed to support a bed of solid particles of the bed of adsorbent (for example, upstream bed for circulation by gravity);
liquid distributing and collecting inserts disposed between the upper screen and a lower screen; and
the lower screen,
   wherein the metal frame is supported on a first side by the shell (for example a weir plate or a first weir plate of the shell) and is supported on a second side (e.g. only) by:
a main beam disposed diametrically in the shell; and/or
the shell (for example a weir plate of the shell) or a central mast (for example a weir plate of the central mast) disposed in the shell,
   wherein the metal frame comprises at least one third side connecting the first side to the second side, and
   wherein the at least one third side has a height and a thickness which are suitable for providing the mechanical strength of the panel.

Advantageously, the self-supported device makes it possible to eliminate secondary beams without adversely affecting the circulation of the fluid inside the panels (this not being the case notably for the device of FR2961112).

This innovation has several other additional advantages:
improvement in the hydrodynamics, both since it is more advantageous for obstacles to be contacted at the bottom of the bed and since the surface area contacted by the main fluid is smaller;
reduction in the mounting time, since the beams are not installed;
reduction in the mass of metal to be utilized, since the increase in height of a beam is broadly advantageous for the reduction in deflection;

improvement in the homogeneity of the adsorbent solid which is charged. In the current technology, the sieve is charged by Catapac® and falls from top to bottom. Underneath the beams, charging is less effective because the dense charging effect does not occur and the feedstock only gets to the sieve after the bottom of the beams is reached, having bounced off an obstacle. This results in less dense charging below the beams than elsewhere, this reducing the quality of the hydrodynamics and the amount of sieve that is charged.

According to one or more embodiments, the height and the thickness of the third side of the metal frame are selected to ensure a deflection of less than 15 mm, preferably less than 10 mm, very preferably less than 7 mm.

According to one or more embodiments, the plate comprises between 12 and 16 panels.

According to one or more embodiments, the column comprises N beds of adsorbent separated by n plates, wherein the number N of beds of adsorbent and the number n of plates are identical and between 4 and 24, preferably between 8 and 19, very preferably between 12 and 15.

According to one or more embodiments, the at least one third side has a height between 300 mm and 1200 mm, preferably between 500 mm and 800 mm.

According to one or more embodiments, the at least one third side has a thickness between 10 mm and 100 mm, preferably between 20 mm and 50 mm.

According to one or more embodiments, the metal frame has a length L between 0.5 m and 5 m.

According to one or more embodiments, the metal frame has a width I between 0.4 m and 2 m, preferably between 0.8 and 1.4 m.

According to one or more embodiments, a first end of the at least one third side is supported by the shell and the second end of the at least one third side is supported by the main beam and/or the central mast, or the shell.

According to one or more embodiments, the frame comprises at least one stiffener that is disposed above the upper screen and connects two opposite third sides of the metal frame.

According to one or more embodiments, the frame comprises from 1 to 3 stiffeners.

According to one or more embodiments, the height of the stiffeners is between 20 and 1200 mm and preferably between 500 and 800 mm.

According to one or more embodiments, the distributing and collecting inserts comprise the following in the direction of flow of the main fluid:
a collector (or collecting zone) designed to collect the main fluid leaving the upstream bed of adsorbent;
a separating sheet, separating the collector from a distributor and comprising at least one outlet opening for sending the main fluid from the collector towards the distributor (or distribution zone); and
the distributor designed to distribute the main fluid across the downstream bed of adsorbent,
   the panel moreover comprising an injection-withdrawal tank which is adjacent to the separating sheet and disposed in a substantially central position of the panel, the injection-withdrawal tank being designed to collect main fluid and/or inject a secondary fluid and to mix the secondary fluid with the main fluid.

According to one or more embodiments, a panel has a substantially triangular (3 sides) or trapezoidal (4 sides) shape as seen in the direction of flow of the main fluid.

According to one or more embodiments, the third sides of the panels of a plate are parallel or concentric or radial.

According to one or more embodiments, the first side and/or the second side of a panel is at least partially in the form of a circular arc.

According to one or more embodiments, the first side and the second side of a panel together form a circular arc.

According to a second aspect, the present invention can be defined as a simulated moving bed separation process, comprising the following steps: feeding a feedstock and a desorbent to at least one column according to the first aspect, and withdrawing at least one extract and at least one raffinate from the column, the feed and withdrawal points in the plates of the column being shifted over time by an amount corresponding to one bed of adsorbent with a changeover period and determining a plurality of operating zones of the column, and notably the following main zones:
by definition, each of the operating zones is denoted by a number:
   zone I for desorption of a product to be separated (e.g. para-xylene) is between the injection of the desorbent and the withdrawal of the extract;
   zone II for desorption of impurities (for example, isomers of the product to be separated) is between the withdrawal of the extract and the injection of the feedstock;
   zone III for adsorption of the product to be separated is between the injection of the feedstock and the withdrawal of the raffinate; and
   zone IV is between the withdrawal of raffinate and the injection of desorbent,
in which process the beds of adsorbent are distributed in zones I to IV in configurations referred to as configurations of the a/b/c/d type, that is to say the distribution of the beds is as follows:
   a is the number of beds in zone I;
   b is the number of beds in zone II;
   c is the number of beds in zone III; and
   d is the number of beds in zone IV,
in which process:

a=(t*0.2)*(1±0.2);

b=(t*0.4)*(1±0.2);

c=(t*0.27)*(1±0.2); and

d=(t*0.13)*(1±0.2), or

a=(t*0.17)*(1±0.2);

b=(t*0.42)*(1±0.2);

c=(t*0.25)*(1±0.2); and

d=(t*0.17)*(1±0.2),
in which process t is a natural integer between 6 and 24, preferably between 8 and 19, very preferably between 12 and 15.

According to one or more embodiments:
the feedstock comprises a mixture of aromatics containing 8 carbon atoms; and/or
the desorbent is chosen from the group made up of one or more isomers of diethylbenzene and toluene, preferably the desorbent is para-diethylbenzene or toluene, very preferably the desorbent is para-diethylbenzene; and/or
the adsorbent used comprises or consists of a faujasite chosen from the group consisting of BaX, BakX and BaLSX.

According to one or more embodiments:
the temperature in the beds of adsorbent is between 140 °C and 189 °C, preferably between 155 °C and 185 °C, very preferably between 170 °C and 180 °C; and/or
the pressure in the beds of adsorbent is between 1 MPa and 10 MPa, preferably between 2 MPa and 4 MPa, very preferably between 2 MPa and 3 MPa; and/or
the changeover period is between 30 seconds and 100 seconds, preferably between 40 seconds and 80 seconds; and/or
the superficial velocity between the beds is between 0.2 cm/s and 2.5 cm/s and preferably between 0.5 cm/s and 2 cm/s.

Other features and advantages of the invention according to the aforementioned aspects will become apparent upon reading the following description and non-limiting exemplary embodiments, with reference to the appended figures described below.

### LIST OF FIGURES

The FIG. 1 shows a view in vertical section of a portion of multistage column comprising beds of adsorbent Ai separated by plates Pi.

The FIG. 2 shows a view from above of an exposed beam structure of a reference multistage column, the exposed beam structure comprising a main beam and 12 secondary beams.

The FIG. 3 shows a view from above of a plate disposed on the exposed beam structure of FIG. 2, the plate comprising 16 meridian panels.

The FIG. 4 shows a view from above of a plate disposed on the exposed beam structure of the shell of the multistage column of FIG. 2, the plate comprising 16 radial panels.

The FIG. 5 shows a 3D view of a reference meridian panel disposed on 2 secondary beams.

The FIG. 6 shows a view in vertical section of a portion of a multistage column utilizing a flow of fluid passing through a bed of adsorbent disposed between two reference meridian panels supported by secondary beams.

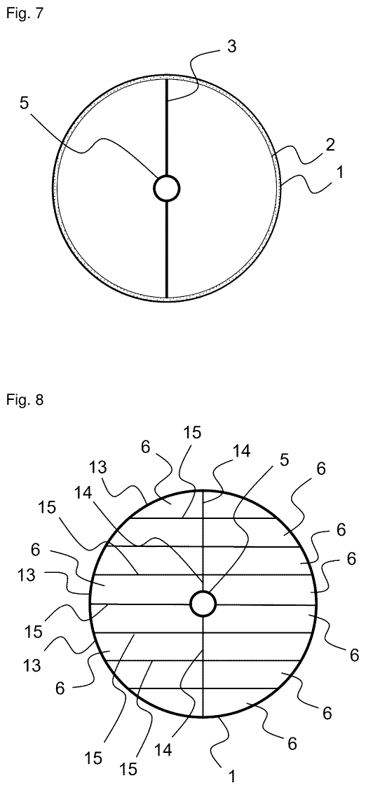

The FIG. 7 shows a view from above of an exposed beam structure of a multistage column according to the present invention, the exposed beam structure comprising only a main beam and no secondary beams.

The FIG. 8 shows a view from above of a plate disposed on the exposed beam structure of FIG. 7, the plate comprising 16 meridian panels according to the present invention.

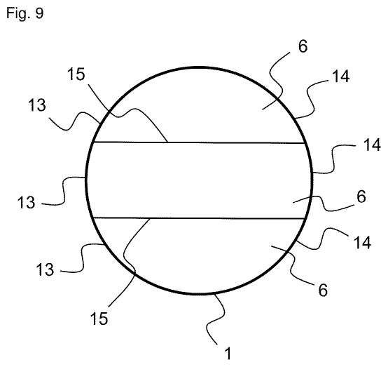

The FIG. 9 shows a view from above of a plate disposed on a radial weir plate of the internal wall of the shell of a multistage column according to the present invention, the shell not having any primary or secondary beam.

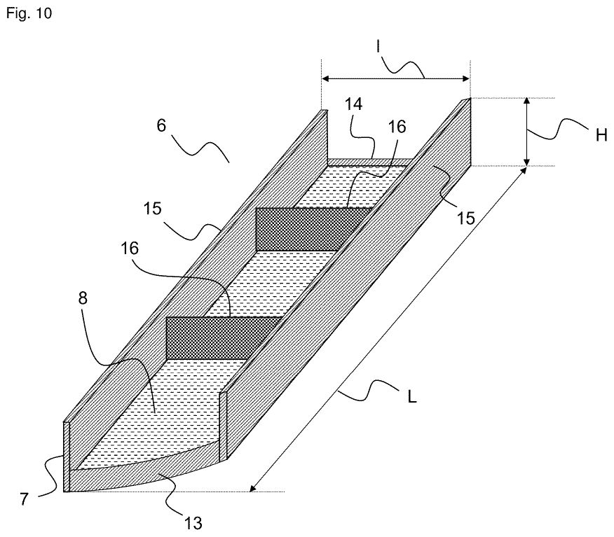

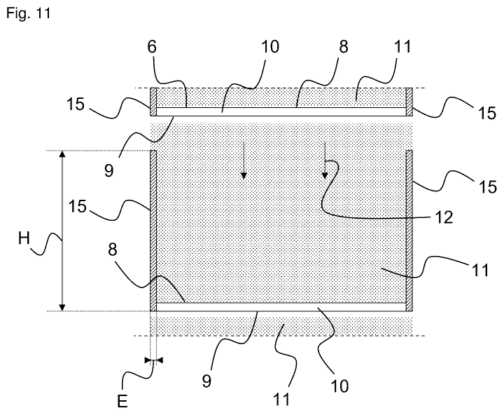

The FIG. 10 shows a 3D view of a meridian panel according to the present invention.

The FIG. 11 shows a view in vertical section of a portion of a multistage column utilizing a flow of fluid passing through a bed of adsorbent disposed between two meridian panels according to the present invention.

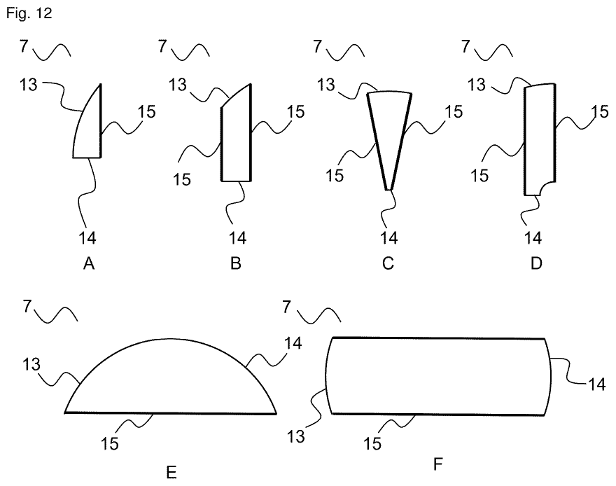

The FIG. 12 shows a view from above of examples of metal frames for meridian panels and an angular-sector panel according to the present invention.

### DETAILED DESCRIPTION OF THE INVENTION

Embodiments of the devices and processes according to the aforementioned aspects will now be described in detail. The following detailed description discloses numerous specific details in order to provide a deeper understanding of the devices and processes. However, it will be apparent to those skilled in the art that the devices and processes can be utilized without these specific details. In other cases, well-known features have not been described in detail in order to avoid unnecessarily complicating the description.

In the present description, the term “to comprise” is synonymous with (means the same thing as) “to include” and “to contain”, and is inclusive or open-ended and does not exclude other elements that are not stated. It will be understood that the term “to comprise” includes the exclusive and closed term “to consist”. In addition, in the present description, the terms “essentially” or “substantially” correspond to an approximation of ±10%, preferably of ±5%, very preferably of ±2%. For example, an element disposed substantially at a certain position of a panel may be disposed in the panel with an approximation of ±10%, preferably ±5%, with respect to the width or the height of the panel.

According to the first aspect, the invention can be defined as a simulated moving bed separation column comprising a shell comprising a plurality of beds of adsorbent separated by a plurality of plates, each plate comprising a plurality of panels referred to as self-supported panels, each panel being designed to collect a main fluid from an upstream bed of adsorbent and to supply the main fluid to a downstream bed of adsorbent. In the present description, the term “self-supported” means that the mechanical strength of the panel is sufficient to dispense with secondary beams, that is to say to provide a maximum deflection of 10 mm, irrespective of the location of the panel.

An SMB separation unit comprises at least one separation column divided into N beds of adsorbent separated by n plates (defining zones between the beds), each plate itself being able to be divided into a plurality of panels. Preferably, the number N of beds of adsorbent and the number n of plates are identical and are between 4 and 24, and preferably between 8 and 19, very preferably between 12 and 15.

The division of the plate Pi into panels is known from the prior art. The two most common types of division are division into meridian panels and division into panels corresponding to angular sectors. The meridian panels correspond to divisions of the plate Pi into elements that are mutually parallel and contiguous so as to ensure complete coverage of the horizontal cross section of the plate. The meridian panels are oriented along a diameter of said plate, and preferably have substantially the same width. According to one or more embodiments, each plate is divided into between 4 and 24 panels, preferably between 12 and 16 panels. The panels are preferably meridian panels.

### The Device

In the present description, the simulated moving bed separation column according to the present invention comprises the same features as the reference column described above with reference to FIGS. 1 to 6, apart from the differences described below.

With reference to FIG. 7, the simulated moving bed separation column according to the present invention comprises a shell 1 in which the mechanical strength of the panels can be provided by one or more weir plates 2 disposed radially (perpendicularly to the direction of flow 12 of the main fluid) on the internal wall of the shell 1.

According to one or more embodiments, the weir plate 2 of the shell 1 is continuous, that is to say annular. According to one or more embodiments, the shell comprises a plurality of discontinuous weir plates.

With reference to FIG. 7, according to one or more embodiments, the simulated moving bed separation column according to the present invention moreover comprises a main beam 3 connecting two radially opposite sides of the shell 1 (disposed perpendicularly to the direction of flow 12 of the main fluid) and/or a central mast 5 (disposed parallel to the direction of flow 12 of the main fluid, for example vertical mast), for example in the form of a tube, the main beam 3 and/or the central mast 5 being designed to (directly) support a plurality of panels 6, referred to as self-supported panels.

With reference to FIG. 8, a panel 6 comprises a metal frame with 3 or 4 sides that are parallel to the direction of flow 12 of the main fluid. According to one or more embodiments, the sides of the metal frame are solid sheets. According to the invention, a first side 13 of the metal frame is disposed on one or more weir plates 2 (for example an annular weir plate) of the shell 1, and a second side 14 of the metal frame is disposed on the main beam 3 and optionally on the central mast 5. According to one or more embodiments, the second side 14 of the metal frame is disposed (only) on the central mast 5, for example when the panels 6 are disposed around the central mast 5 in a radial cutout (disposition of the panels similar to that of FIG. 4).

With reference to FIG. 9, the first side 13 of the metal frame is disposed on one or more weir plates 2 (for example an annular weir plate) of the shell 1, and the second side 14 of the metal frame is disposed on one or more weir plates 2 (for example the annular weir plate) of the shell 1. Advantageously, when the simulated moving bed separation column does not comprise a main beam or central mast, for example when the diameter of the shell is less than 5 m, the two sides 13 and 14 of the panels 6 may rest against the shell 1.

With reference to FIG. 10 and FIG. 11, the metal frame 7 of the panel 6 serves to hold the elements of the panel 6 (screens and inserts) together, and notably comprises:
an upper screen 8 or any other equivalent device (for example perforated sheet) for supporting the bed of solid particles and ensuring the passage of the main fluid;
a lower screen 9 or any other equivalent device (for example perforated sheet) for ensuring the passage of the main fluid, and
distributing and collecting inserts 10 situated between said screens.

With reference to FIGS. 8 to 12, the first side 13 and the second side 14 of the metal frame 7 are connected to one another by means of at least one third side (15), and the at least one third side (15) has a height (H) and a thickness (E) which are suitable for providing the mechanical strength of the panel, this making it possible to eliminate secondary beams without adversely affecting the circulation of the fluid inside the panels.

With reference to FIG. 10 and FIG. 11, according to one or more embodiments, the height H and the thickness E of the third side 15 of the metal frame 7 are selected to ensure a deflection of less than 15 mm, preferably less than 10 mm, very preferably less than 7 mm.

According to one or more embodiments, the at least one third side 15 has a height H between 300 mm and 1200 mm, preferably between 500 mm and 800 mm. According to one or more embodiments, the height H of the third side 15 of the metal frame 7 is substantially equal to the height of the upstream bed of adsorbent (for example ±10%, preferably ±5%, very preferably ±2%).

According to one or more embodiments, the at least one third side 15 has a thickness E between 10 mm and 100 mm, preferably between 20 mm and 50 mm.

According to one or more embodiments, the metal frame 7 (for example the at least one third side 15) has a maximum length L between 0.5 m and 5 m.

According to one or more embodiments, the metal frame 7 has a maximum width I (perpendicular to the third side 15) between 0.4 m and 2 m, preferably between 0.8 and 1.4 m.

With reference to FIG. 10, according to one or more embodiments, the frame comprises one or more stiffeners 16 (metal elements) which are disposed above the upper screen 8 and are connected to at least one third side 15. Advantageously, the at least one stiffener 16 is adapted to be immersed in the adsorbent bed and makes it possible to avoid buckling of the at least one third side 15. According to one or more embodiments, the at least one stiffener 16 is connected to two opposite third sides 15 of the metal frame 7. According to one or more embodiments, the third sides 15 of the panels 6 of a plate are parallel or concentric or radial.

According to one or more embodiments, the at least one stiffener 16 forms an angle between 60° and 90° with the at least one third side 15. According to one or more embodiments, the at least one stiffener 16 is substantially perpendicular to the at least one third side 15.

According to one or more embodiments, the at least one stiffener 16 forms an angle between 65° and 85°, preferably between 75° and 80°, with the at least one third side 15.

According to one or more embodiments, the at least one stiffener 16 has a height (parallel to the direction of flow 12 of the main fluid) between 20 mm and 1200 mm and preferably between 500 and 800 mm. According to one or more embodiments, the at least one stiffener 16 has a height of at least 50% of the height H of the at least one third side 15. According to one or more embodiments, the frame comprises from 1 to 3 stiffeners 16.

With reference to FIG. 12, the metal frame 7 (as seen in the direction of flow 12 of the main fluid) may have substantially the shape of a triangle A, a circular arc B or a trapezium C.

According to one or more embodiments, the metal frame 7 comprises a recess, as shown for the circular arc B and the trapezium D, to allow the passage of the central mast 5.

It should be understood that a metal frame may have a different shape. For example, when the shell does not comprise a main beam 3 or a central mast 5 (see FIG. 9), the metal frame 7 may have the shape of a circular segment. A first example of a circular segment E shows part of a disc cut off from the rest of the disc by a chord (intersecting straight line), wherein the first side 13 and the second side 14 together form the circular arc, and the third side 15 forms the chord. A second example of a circular segment F shows part of a disc cut off from the rest of the disc by two mutually parallel chords, wherein the first side 13 and the second side 14 each form a circular arc, and two third sides 15 form the chords. According to the present description, a circular segment makes up that part of a disc that is between an intersecting straight line and an arc (circular segment E), or between two intersecting straight lines (circular segment F).

The distributing and collecting inserts 10 generally comprise the following, in the direction of flow 12 of the main fluid:
a collector or collecting zone designed to collect the main fluid leaving an upstream bed of adsorbent;
a separating sheet, separating the collector from the distributor;
a distributor or distribution zone designed to distribute the main fluid collected on its own or in a mixture with a secondary fluid across the downstream bed of adsorbent,
   the upper screen, the collector, the separating sheet, the distributor and the lower screen extending from the first side 13 to the second side 14.

The distributing and collecting inserts 10 moreover comprise an injection-withdrawal tank designed to extract main fluid collected by the collector or inject a secondary fluid so as to mix said secondary fluid with the main fluid. The injection-withdrawal tank is adjacent to the separating sheet and is disposed in a central position of the panel 3, that is to say a position situated substantially in the vertical central axis of the panel. The vertical central axis of the panel is a transverse axis of the panel, that is to say an axis which is parallel to the direction of flow 12 of the fluid and orthogonal to the plane formed by the separating sheet.

Advantageously, the upper screen and the separating sheet together form the collector (collection zone) designed to direct the main fluid towards the injection-withdrawal tank.

Advantageously, the separating sheet comprises two lateral parts situated on either side of the injection-withdrawal tank, that is to say the injection-withdrawal tank separates the separating sheet into two lateral parts.

Advantageously, the separating sheet comprises at least one, and preferably at least two, outlet openings, preferably disposed close to or adjacent to the injection-withdrawal tank and being designed to send the main fluid from the collector towards the distributor. Preferably, at least one outlet opening is disposed on either side of the injection-withdrawal tank.

Depending on the operating mode of the panel, the main fluid can thus be collected in the injection-withdrawal tank or mixed with a secondary fluid leaving the injection-withdrawal tank. The main fluid and the secondary fluid that are mixed in this way are redistributed to the downstream bed of adsorbent Ai when they enter the distributor.

Advantageously, the lower screen and the separating sheet together form the distributor (distribution zone) for directing the main fluid collected on its own or in a mixture with a secondary fluid towards the downstream bed of adsorbent Ai.

### The Process

The invention can also be defined as an SMB process using an SMB separation unit according to the invention, in which the feedstock to be separated is any mixture of compounds, such as aromatics having from 7 to 9 carbon atoms, a mixture of normal and iso-paraffins, or a mixture of normal and iso-olefins.

The invention therefore also relates to an SMB separation process using at least one separation column 1 divided into N beds of adsorbent Ai separated by n plates Pi comprising a plurality of panels 3 according to the invention.

In the remainder of the text, reference is made to a step in order to denote an operation or a group of similar operations carried out on a given stream at a certain point of the process.

The process is described in terms of its various steps taken in the order of flow of the streams or products.

The SMB separation process comprises the following steps: feeding at least one feedstock and a desorbent to the column 1, and withdrawing at least one extract and at least one raffinate from the column 1, said column 1 comprising one or more beds of an adsorbent solid Ai that are interconnected in a closed circuit (that is to say the last bed of the last adsorbent is designed to send the flow to circulate in the first bed of the first adsorbent) and separated by plates Pi according to the invention, the feed and withdrawal points in the plates of the column being shifted over time by an amount corresponding to one bed of adsorbent with a changeover period (denoted ST) and determining a plurality of operating zones of the SMB device, and notably the following main zones, by definition denoted by a number:
zone I for desorption of the product (of interest) to be separated is between the injection of the desorbent and the withdrawal of the extract;
zone II for desorption of impurities (for example, isomers of the product to be separated) is between the withdrawal of the extract and the injection of the feedstock;
zone III for adsorption of the product to be separated is between the injection of the feedstock and the withdrawal of the raffinate; and
zone IV is between the withdrawal of raffinate and the injection of desorbent.

According to one or more embodiments, the beds of adsorbent are distributed in zones I to IV in configurations referred to as configurations of the a/b/c/d type, that is to say the distribution of the beds is as follows:
a is the number of beds in zone I;
b is the number of beds in zone II;
c is the number of beds in zone III; and
d is the number of beds in zone IV.

According to one or more embodiments:

a=(t*0.2)*(1±0.2);

b=(t*0.4)*(1±0.2);

c=(t*0.27)*(1±0.2); and

d=(t*0.13)*(1±0.2), and

in which t is a natural integer between 6 and 24, preferably between 8 and 19 (e.g. between 12 and 15).

According to one or more embodiments:

a=(t*0.17)*(1±0.2);

b=(t*0.42)*(1±0.2);

c=(t*0.25)*(1±0.2); and

d=(t*0.17)*(1±0.2), and

in which t is a natural integer between 6 and 24, preferably between 8 and 19, very preferably between 12 and 15 (e.g. 12 or 15).

According to one or more embodiments, the desorbent is chosen from the group made up of one or more isomers of diethylbenzene and toluene. According to one or more embodiments, the desorbent is para-diethylbenzene or toluene. According to one or more embodiments, the desorbent is para-diethylbenzene.

According to one or more embodiments, the adsorbent used comprises/consists of a faujasite chosen from the group consisting of BaX, BaKX and BaLSX.

According to one or more embodiments, the feedstock is a mixture of essentially C8 aromatic compounds (e.g. xylenes and ethylbenzene). According to one or more embodiments, the mixture comprises at least 95%, preferably at least 97% (e.g. at least 99%), essentially C8 aromatic compounds. According to one or more embodiments, the feedstock comprises at least 15% by weight para-xylene and/or 30% by weight of meta-xylene relative to the total weight of the feedstock.

One example of an SMB separation process of great industrial importance concerns the separation of C8 aromatic fractions in order to produce para-xylene of commercial purity, typically at a purity of at least 99.7% by weight, and a raffinate rich in ethylbenzene, ortho-xylene and meta-xylene.

The extract produced contains desorbent, para-xylene and possibly traces of isomers (para-xylene purity greater than 98%, preferably greater than 99.7%). This extract can be treated in order to separate the desorbent (for example by distillation) and may optionally be purified by crystallization to increase the purity of the para-xylene.

According to one or more embodiments, the temperature in the beds of adsorbent is between 140 °C and 189 °C and preferably between 155 °C and 185 °C, particularly preferably between 170 °C and 180 °C.

The pressure is adjusted so that the liquid phase is maintained at all points of the process according to the invention. According to one or more embodiments, the pressure in the beds of adsorbent is between 1 MPa and 10 MPa, preferably between 2 MPa and 4 MPa, preferably between 2 MPa and 3 MPa.

According to one or more embodiments, the changeover period ST (period of time between two successive changeovers between feeds/extractions) employed is between 30 seconds and 100 seconds. Preferably, the changeover period ST employed is between 40 seconds and 80 seconds (for example 60±10 seconds).

According to one or more embodiments, the superficial velocity between the beds is between 0.2 cm/s and 2.5 cm/s and preferably between 0.5 cm/s and 2 cm/s.

### EXAMPLES

### Example 1: Dimensioning

The deflection of a metal sheet is inversely proportional to its area moment of inertia I_{qz}, which, for a sheet of rectangular cross section of height (H) and thickness (E), is as defined below in the mathematical formula Math 1.

$${I}_{q z} = \frac{E {H}^{3}}{12} \qquad Math 1$$

This moment demonstrates that the height has a much greater impact on the vertical deformation than the thickness of the sheet.

As a result, it is possible to compare a reference device and a device according to the invention in which the height of the third side (15) of the metal frame (7) has been increased to the height of the bed. Table 1 below proposes this comparison with consideration of an identical deflection in both cases, thus with iso-deformation. The results show that the device according to the invention makes it possible to reduce the mass of metal involved by 70%. The surface area that is covered by the fluid circulation may serve as hydrodynamics indicator, and also shows a significant improvement.

TABLE 1

| Third side 15 | Reference | Invention |
|---|---|---|
| Height H (mm) | 450 | 800 |
| Thickness E (mm) | 100 | 17 |
| Length (mm) | 4000 | 4000 |
| Volume (m³) | 0.18 | 0.05 |
| Area moment of inertia I_{qz} (mm⁴) | 7.6 × 10⁸ | 7.6 × 10⁸ |
| Surface area covered (m²) | 0.40 | 0.07 |

### Example 2: Hydrodynamics

The hydrodynamics of a reference device and a device according to the invention is evaluated by a CFD simulation and the results are compared in terms of the Péclet number. It is a dimensionless number illustrating the ratio of convection to axial dispersion. The higher this number is, the greater the plug-flow effect is and the better the performance of the process is. The Péclet number is calculated according to the following formula, in which μ is the first moment and σ is the central second moment. The moments are obtained by calculating the propagation of the moments in the bed in accordance with the methodology described by Liu et al (see AlChE Journal, vol. 56, issue 10, pages 2561-2572), as defined below in the mathematical formula Math 2.

$$P e = 2 \times \frac{{μ}^{2}}{{σ}^{2}} \qquad Math 2$$

Table 2 below illustrates the improvements obtained for the Péclet number. The case according to the invention was carried out with a third side 15 forming the entire height of the bed.

TABLE 2

| | Reference | Invention |
|---|---|---|
| Third side 15 | | |
| Height H (mm) | 400 | 1200 |
| Thickness E (mm) | 80 | 30 |
| Length (mm) | 4250 | 4250 |
| Hydrodynamics | | |
| Superficial velocity (cm/s) | 1.2 | 1.2 |
| Height of bed of adsorbent (m) | 1.2 | 1.2 |
| Péclet number | 142 | 193 |

### Example 3: Improvement in Mounting Time

The current technology suffers from lengthy plate mounting/dismounting times which adversely affect the operating time for the entire aromatic complex. Stoppages to change sieves can take months. The innovation presented makes it possible to dispense with the assembly of secondary beams. Moreover, the step of stabilizing the panels on the beams no longer takes place. As a result, work inside the towers is reduced. The table below proposes an estimation of the improvement in time per bed of adsorbent, which is around 30%.

TABLE 3

| Installation duration | Reference | Invention |
|---|---|---|
| Installation of main beams | 1 h | 1 h |
| Installation of secondary beams | 7 h | 0 |
| Installation of panels | 8 h | 8 h |
| Stabilization of panels on the beams | 8 h | 0 |
| Sealing | 8 h | 8 h |
| Installation of pipework + charging of sieve | 16 h | 16 h |
| Total | 48 h | 33 h |

1. Simulated moving bed separation column comprising a shell (1) comprising a plurality of beds of adsorbent separated by a plurality of plates, each plate comprising a plurality of panels (6) referred to as self-supported panels, each panel (6) being designed to collect a main fluid from an upstream bed of adsorbent (11) and to supply the main fluid to a downstream bed of adsorbent (11), each panel (6) comprising a metal frame (7) on multiple sides, the following being disposed said metal frame (7):
an upper screen (8) designed to support a bed of solid particles of the upstream bed of adsorbent (11);
liquid distributing and collecting inserts (10) disposed between the upper screen (8) and a lower screen (9); and
the lower screen (9),
wherein the metal frame (7) is supported on a first side (13) by the shell (1) and is supported on a second side (14) by:
a main beam (3) disposed diametrically in the shell (1); and/or
the shell (1) or a central mast (5) disposed in the shell (1),
wherein the metal frame (7) comprises at least one third side (15) connecting the first side (13) to the second side (14), and
wherein the at least one third side (15) has a height (H) and a thickness (E) which are suitable for providing the mechanical strength of the panel.

2. Column according to claim 1, wherein the height (H) and the thickness (E) of the third side (15) of the metal frame (7) are selected to ensure a deflection of less than 15 mm, preferably less than 10 mm, very preferably less than 7 mm.

3. Column according to claim 1, comprising N beds of adsorbent separated by n plates, wherein the number N of beds of adsorbent and the number n of plates are identical and between 4 and 24, preferably between 8 and 19, very preferably between 12 and 15, and/or wherein each plate comprises between 12 and 16 panels (6).

4. Column according to claim 1, wherein the at least one third side (15) has a height (H) between 300 mm and 1200 mm, preferably between 500 mm and 800 mm.

5. Column according to claim 1, wherein the at least one third side (15) has a thickness (E) between 10 mm and 100 mm, preferably between 20 mm and 50 mm.

6. Column according to claim 1, wherein the metal frame (7) has a length L between 0.5 m and 5 m, and/or the metal frame (7) has a width 1 between 0.4 m and 2 m, preferably between 0.8 and 1.4 m.

7. Column according to claim 1, wherein a first end of the at least one third side (15) is supported by the shell (1) and the second end of the at least one third side (15) is supported by the main beam (3) and/or the central mast (5), or the shell (1).

8. Column according to claim 1, wherein the frame comprises at least one stiffener (16) that is disposed above the upper screen (8) and connects two opposite third sides (15) of the metal frame (7).

9. Column according to claim 8, wherein the frame comprises from 1 to 3 stiffeners (16), and/or the height of the stiffener (16) is between 20 and 1200 mm and preferably between 500 and 800 mm.

10. Column according to claim 1, wherein the distributing and collecting inserts comprise the following in the direction of flow (12) of the main fluid:
a collector designed to collect the main fluid leaving the upstream bed of adsorbent;
a separating sheet, separating the collector from a distributor and comprising at least one outlet opening for sending the main fluid from the collector towards the distributor; and
the distributor designed to distribute the main fluid across the downstream bed of adsorbent,
the panel moreover comprising an injection-withdrawal tank which is adjacent to the separating sheet and disposed in a substantially central position of the panel, the injection-withdrawal tank being designed to collect main fluid and/or inject a secondary fluid and to mix the secondary fluid with the main fluid.

11. Column according to claim 1, wherein a panel (6) is substantially triangular or trapezoidal as seen in the direction of flow (12) of the main fluid.

12. Column according to claim 1, wherein the third sides (15) of the panels (6) of a plate are parallel or concentric or radial.

13. Column according to claim 1, wherein the first side (13) and/or the second side (14) of a panel (6) is at least partially in the form of a circular arc.

14. Column according to claim 1, wherein the first side (13) and the second side (14) of a panel (6) together form a circular arc.

15. Simulated moving bed separation process, comprising the following steps: feeding a feedstock and a desorbent to at least one column according to claim 1, and withdrawing at least one extract and at least one raffinate from the column, the feed and withdrawal points in the plates of the column being shifted over time by an amount corresponding to one bed of adsorbent with a changeover period and determining a plurality of operating zones of the column, and notably the following main zones: by definition, each of the operating zones is denoted by a number:
zone I for desorption of a product to be separated is between the injection of the desorbent and the withdrawal of the extract;
zone II for desorption of impurities is between the withdrawal of the extract and the injection of the feedstock;
zone III for adsorption of the product to be separated is between the injection of the feedstock and the withdrawal of the raffinate; and
zone IV is between the withdrawal of raffinate and the injection of desorbent;
in which process the beds of adsorbent are distributed in zones I to IV in configurations referred to as configurations of the a/b/c/d type, that is to say the distribution of the beds is as follows:
a is the number of beds in zone I;
b is the number of beds in zone II;
c is the number of beds in zone III; and
d is the number of beds in zone IV,
in which process:
a=(t*0.2)*(1±0.2);
b=(t*0.4)*(1±0.2);
c=(t*0.27)*(1±0.2); and
d=(t*0.13)*(1±0.2), or
a=(t*0.17)*(1±0.2);
b=(t*0.42)*(1±0.2);
c=(t*0.25)*(1±0.2); and
d=(t*0.17)*(1±0.2),
in which process t is a natural integer between 6 and 24, preferably between 8 and 19, very preferably between 12 and 15.
